(19) United States
(12) Patent Application Publication
(10) Pub. No.: US 2009/0017503 A1
(43) Pub. Date: Jan. 15, 2009

(54) Method and Apparatus for Saccharide Precipitation From Pretreated Lignocellulosic Materials

(75) Inventors: Yi-Heng Percival Zhang, Blacksburg, VA (US); Lee R. Lynd, Meriden, NH (US)

Correspondence Address: LATHROP & GAGE LC, 4845 PEARL EAST CIRCLE, SUITE 300, BOULDER, CO, 80301, US

(73) Assignee: THE TRUSTEES OF DARTMOUTH COLLEGE, Hanover, NH (US)

(21) Appl. No.: 12/063,011

(22) PCT Filed: Aug. 7, 2006
(86) PCT No.: PCT/US06/30894

§ 371 Date: Sep. 8, 2008

Related U.S. Application Data
(60) Provisional application No. 60/705,985, filed on Aug. 5, 2005.

(51) Int. Cl. C12P 19/00; C07H 1/08

(52) U.S. Cl. 435/72; 536/128

(57) ABSTRACT

A method for separating saccharide components and lignin fractions from a concentrated acid treated lignocellulosic biomass is disclosed. The method involves precipitating the saccharide components by adding an organic solvent to the biomass slurry. The acid may then be recovered, for example, by filtration or by countercurrent washing and the organic solvent may be flashed and recycled. During acid recovery and organic recovery steps, two main lignocellulose components (hemicellulose and lignin) as well as minor components such as acetic acid are separated as well. The method decreases the amount of cellulase required for hydrolysis, increases hydrolysis rates, reduces formation of inhibitor molecules, increase sugar yields, produces high value by-products such as high quality lignin and hemicellulose, and decreases energy and equipment costs.

### RELATED APPLICATIONS

This application claims the benefit of priority to U.S. Application No. 60/705,985, filed Aug. 5, 2005, which is incorporated herein by reference.

### GOVERNMENT INTERESTS

The United States Government may have certain rights in the present invention as research relevant to its development was funded by United States Department of Energy (DOE) contract number DE FG02-02ER15350 and by National Institute of Standards and Technology (NIST) contract number 60NANB1D0064.

### BACKGROUND

1. Field of the Invention

The present invention pertains to the field of biomass processing to produce fuels, chemicals and other useful products and, more specifically, to isolating saccharide components and lignin from an acidified or saccharified lignocellulosic biomass slurry. Isolation of the saccharide component leads to improved sugar yields, greater overall efficiency, and potential economic profitability and flexibility.

2. Description of the Related Art

Lignocellulosic materials, or biomass, (e.g. wood and solid wastes), have been used as source materials to generate saccharides, which in turn may be used to produce ethanol and other products. Ethanol has a number of industrial and fuel uses. Of particular interest is the use of ethanol as a gasoline additive that boosts octane, reduces pollution, and partially replaces gasoline in fuel mixtures. It has been proposed to eliminate gasoline almost completely from fuel and to burn ethanol in high concentrations.

Conversion of lignocellulosic biomass into renewable fuels and chemicals often involves treatment of the biomass with concentrated acid. The concentrated phosphoric acid breaks not only lignin seals, and connections among cellulose, hemicellulose, and lignin, but also hydrogen bonds among hemicellulose and cellulose chains, i.e. polysaccharides. Further, the concentrated acid weakly degrades the glycosidic bonds formed between the monomeric units. The saccharides are then separated from the acid before they can be converted into alcohols and other products.

A number of conventional methods have been used to separate acid-saccharide solutions in bioconversion processes. For example, the acid-saccharide solution may be passed through an activated charcoal filter that retains the saccharides. The adsorbed saccharides may subsequently be eluted from the charcoal filter by washing with heated alcohol. However, this method for separating acid and saccharides requires the alcohol to be evaporated from the resulting saccharide solution before fermentation, which adds an additional step requiring energy input. Ion exchange resins may also be used to separate the acid and saccharides. The saccharides are adsorbed on the strongly acidic resin giving an acid containing stream which can be recycled. The adsorbed saccharides are then recovered by rinsing the resin with pure water. Strong acid cation exchange resins cost about $100/ft³ and their regenerative capacity diminishes with each cycle. A third approach is to separate the acid and saccharides by extraction that removes the acid from the aqueous solution. The separation may be carried out, for example, on a Karr reciprocating-plate extraction column.

The specialized equipment and high energy costs of the acid-saccharide separation techniques described above have led to the development of alternative hydrolysis processes. Current research is largely focused on enzymatic hydrolysis, where biomass is pretreated using dilute acid at elevated temperatures and pressures, or by steam explosion, to open the structure of the lignocellulosic material. Enzymes are then added to the pretreated material to hydrolyze cellulose and hemicellulose. However, enzymatic hydrolysis is a fairly slow process and the cost of enzymes is high, especially where lignin (a recalcitrant biomass component) binds and inactivates these enzymes. Some biomass with a high lignin content, e.g. softwood, has been largely avoided as a feedstock for bioconversion due to lignin-blocking of the enzymatic hydrolysis process.

### SUMMARY

The present invention advances the art and overcomes the problems outlined above by providing an efficient method for separating saccharides from acid treated biomass. An organic solvent is used to precipitate saccharides from acidic solution. Acid is then recovered and reused by evaporating or distilling the organic solvent, which preferably has a low boiling point. Among other advantages, the separation and recovery processes described herein lead to high saccharide yields, fast hydrolysis rates, and low capital investment and energy requirements.

In one embodiment, a method for improving a bioconversion process includes combining a biomass with a composition including an acid to provide a biomass slurry and liberate saccharide components thereof, precipitating the saccharide components by adding an organic solvent to the biomass slurry, and removing the acid from the precipitated saccharide components.

According to one embodiment, a method includes redissolving and fermenting the precipitated saccharide in the presence of a sugar-to-ethanol converting microorganism for a period of time and under suitable conditions for producing ethanol.

Still other embodiments pertain to improved processes for producing an organic compound from a lignocellulosic biomass.

### BRIEF DESCRIPTION OF THE DRAWINGS

FIG. 1 is a schematic diagram showing process equipment that may be used according to one embodiment that incorporates saccharide precipitation in a lignocellulose conversion process.

### DETAILED DESCRIPTION

There will now be shown and described a method for increasing process efficiency in making useful products out of lignocellulosic biomass. Efficiency may be gained by the present method which advantageously:
increases sugar yields;
decreases the amount of cellulase required for hydrolysis;
performs pretreatment processes at ambient or modest temperature and pressure;
increases hydrolysis rates;
reduces or avoids formation of inhibitor molecules;
decreases energy and equipments costs associated with chemical separation and solvent recovery; and
allows for isolation of high value by-products.

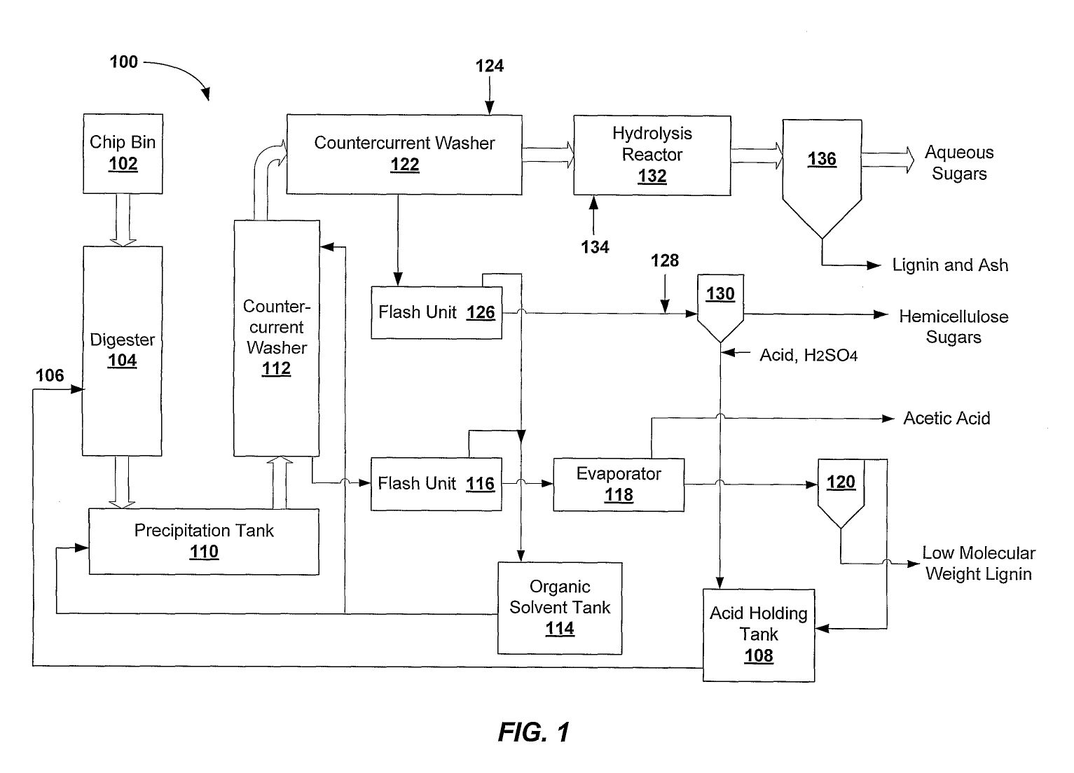

FIG. 1 shows one embodiment of a reactor system 100 that may be used for biomass conversion. Particulate lignocellulosic material from a chip bin 102 is added to a digester 104. The particulate lignocellulosic material may range in size from less than 1 millimeter in diameter to several inches in diameter, and may, for example, have been previously processed by a chopper mill. The particle size is not necessarily critical but hydrolysis generally proceeds faster with a smaller particle size. An economic optimization may be reached between the costs of grinding the lignocellulosic material and the cost advantages of higher throughput. Smaller particle sizes inherently provide more surface area. On the other hand, for a given set of flow conditions, particles that are too small may form a dense mat, which is difficult for fluid to penetrate at an acceptable rate.

It will be appreciated that the lignocellulosic material may be any feedstock that contains cellulose. In various embodiments, the lignocellulosic biomass comprises wood, corn stover, sawdust, bark, leaves, agricultural and forestry residues, grasses such as switchgrass, ruminant digestion products, municipal wastes, paper mill effluent, newspaper, cardboard, or combinations thereof. Reactor system 100 may accept various feedstocks, and any agricultural, industrial, or municipal process that uses or discharges such wastes may be modified to incorporate reactor system 100.

Acid 106, such as phosphoric acid, is added to digester 104 from acid holding tank 108. For example, acid 106 may, for example, be concentrated or diluted to add about 25%, 20%, 15%, 10%, 8%, 6%, 4%, 3%, 2%, 1% or less than 1% water by weight. The term “concentrated acid” may refer to a pure acid (i.e. 0% water), but it is more commonly used to refer to an acidic aqueous solution that is sold commercially as a “concentrated acid” that contains between about 40-99% by weight acid. Examples of such “concentrated acids” include commercially available “concentrated phosphoric acid”, which is typically 14.8 M (85.5% by weight acid), and “concentrated hydrochloric acid”, which is typically 12.1 M (37.2% by weight acid). Digester 104 is typically operated at ambient temperature and pressure, but it may optionally be heated and/or sealed. The slurry within digester 104 is stirred or agitated, for example, by mixing blades, pumps, or bubbling with an inert gas, such as argon or nitrogen. Following an amount of time that is sufficient for acid hydrolysis, which is usually between about one half hour and twelve hours, the slurry from digester 104 is transferred to precipitation tank 110. Precipitation tank 110 may be prefilled with an organic solvent, such as acetone, in an amount ranging from about a 2-100 fold volumetric excess relative to the volume of the slurry. Alternatively, precipitation tank 110 may be empty when the slurry from digester 104 is transferred and organic solvent may be added later, or the slurry and organic solvent may be added to precipitation tank 110 simultaneously. Combining the slurry and organic solvent results in precipitation of highly reactive amorphous saccharides.

Organic solvents useful for effecting precipitation include any organic solvent, or mixture of organic solvents, that substantially reduces the solubility of saccharides in acidic aqueous solution, and especially, for example, low molecular weight, water-miscible solvents such as methanol, ethanol, n-propanol, isopropanol, acetone, other low molecular weight alcohols, glycols or ketones, and combinations thereof. The organic solvent is present in a quantity sufficient to substantially reduce the polarity of the slurry solvent. For example, the organic solvent is usually provided in about a 2-100 fold volumetric excess relative to the volume of the slurry solvent.

Precipitation tank 110 discharges liquid and solid components into a first countercurrent washer 112. Organic solvent, which may come from organic solvent tank 114, is added to the bottom of countercurrent washer 112. Light fractions from the top of countercurrent washer 112 are removed to flash unit 116. Organic solvent from flash unit 116 is recycled to organic solvent tank 114 and acetic acid, a high value by-product, is collected from an evaporator 118. Liquids and solids remaining after evaporation of acetic acid are transferred to a vortex separator 120. Low molecular weight lignin, a high value by-product, is recovered from vortex separator 120 and acid is recycled to acid holding tank 108. The majority of acid that was added to digester 104 is removed and recycled by vortex separator 120.

Heavy fractions within first countercurrent washer 112 are transferred to a second countercurrent washer 122. Hot water 124 is added to the bottom of countercurrent washer 122 to wash precipitated saccharides, that were precipitated in precipitation tank 110 and separated from the majority of acid by flash unit 116. Light fractions from the top of countercurrent washer 122 are transferred to flash unit 126. Organic solvent from flash unit 126 is recycled to organic solvent tank 114 and a lime (CaCO₃) or calcium hydroxide (Ca(OH)₂) solution 128, e.g., one with sufficient lime to impart a pH of about 5 to 7, is added to the effluent of flash unit 126 to neutralize any remaining acid. The neutralized effluent enters vortex separator 130 where hemicellulose sugars in the aqueous phase are separated from precipitated Ca₃(PO₄)₂. After removal of the hemicellulose fraction, the remaining discharge of vortex separator 130 is acidified, for example, with sulfuric acid to convert insoluble Ca₃(PO₄)₂ to weakly soluble CaSO₄, and recycled to acid holding tank 108. Heavy fractions within countercurrent washer 122 (e.g. cellulose and lignin) are transferred to hydrolysis reactor 132 and an enzymatic solution 134 is added. Enzymatic solution 134 contains a hydrolyzing enzyme, for example, cellulase. Alternatively, enzymatic solution 134 contains an inoculum and growth medium including a microorganism capable of saccharifying the slurry for hydrolysis of cellulose by the in vivo production of such enzymes, e.g. Clostridium cellulolyticum, Clostridium thermocellum, Clostridium acetobutylicum. Cellulose prepared by the present instrumentalities may be hydrolyzed using only thermostable endoglucanase. The cellulose does not require exoglucanase and/or glucosidase as is required for conventionally pretreated cellulose.

Hydrolysis reactor 132 may be heated and may be one of a series of such reactor vessels, which may permit continuous batch processing. The residence time in hydrolysis reactor 132 may be from one to three days. Hydrolysis reactor 132 may, for example, be a flow-through reactor in which solids are retained for an interval of time with recycle of fluids, a fluidized bed reactor with fluid recycle, or a stir-tank. Effluent from hydrolysis reactor 132 enters vortex separator 136, where solids such as lignin and ash are removed from the aqueous saccharide solution. The lignin and ash can be burnt to supply energy for reactor 100 or other applications.

The aqueous saccharide solution may be recovered from vortex separator 136 as a final product or it may enter another reactor (not shown) where a second enzymatic solution, which may contain a fermentation microorganism or enzymes useful for the conversion of sugars into alcohols, is added. Useful products, e.g., ethanol, may be distilled from the fermentation broth.

One example of an organism that is useful in converting organic matter to ethanol is Clostridium thermocellum. Other examples of suitable microorganisms that may be used include Fusarium oxysporum and C. cellulolyticum. In addition, such organisms can be used in co-culture with C. thermosaccharolyticum or similar pentose-utilizing organisms such as C. thermohydrosulfuricum and Thermoanaerobacter ethanoliticus. An example of another microorganism that produces enzymes for both hydrolysis and fermentation in a Simultaneous Saccharification and Fermentation process is Saccharomyces cerevisiae.

A variety of suitable growth media for microbial digestion processes are well known in the art. Generally, a suitable growth medium is able to provide the chemical components necessary to maintain metabolic activity and to allow cell growth. One effective growth medium contains the following components per liter of water:

| | |
|---|---|
| protein treated wood | 5.0 g |
| NaH₂PO₄ | 0.3 g |
| K₂ SO | 0.7 g |
| NH₂SO₄ | 1.3 g |
| Yeast extract | 2.0 g |
| Morpholinopropanesulfonic acid (MOPS) | 2.0 g |
| Cysteine Hydrochloride | 0.4 g |
| MgCl₂₆H₂O | 0.2 g |
| CaCl₂₆H₂O | 0.1 g |
| FeSO₄ | 0.1 g |

The medium noted above is set forth by way of example. Other suitable growth media may be used.

It will be appreciated that the equipment shown generally in FIG. 1 may be used or adapted to implement a variety of known processes. The prior processes do not include use of a precipitation step, such as that performed in precipitation tank 110, and may be adapted for such use according to the instrumentalities described herein. The aforementioned use of the precipitation step results in significant cost reductions in the overall process of producing saccharides or fermented organic compounds from lignocellulose by improving recovery and separation processes.

Generally, any lignocellulosic saccharification process may be improved by using an organic solvent to precipitate saccharides, which facilitates separation and fluid recycling. The process may, for example, entail making pulp, making paper, treating effluent from a pulp manufacturing process, treating effluent from a process of making paper, a bioconversion process, a biopolymer process, a protein-binding analytic assay, an enzymatic analytic assay, a waste treatment process, and combinations thereof.

It will be appreciated that numerous modifications to the equipment of FIG. 1 may be made. For example, in an alternate embodiment vortex separator 136 may be incorporated between countercurrent washer 122 and hydrolysis reactor 132. In this arrangement, lignin may be removed prior to enzymatic hydrolysis, and inhibition due to non-productive enzyme binding with lignin may be reduced or avoided.

All references mentioned in this application are incorporated by reference to the same extent as though fully replicated herein.

What is claimed is:

1. A method for improving a bioconversion process, comprising:
combining a biomass with a composition including an acid to provide a biomass slurry and liberate a saccharide component thereof;
precipitating at least part of the saccharide component by adding an organic solvent to the biomass slurry; and
removing the acid from the precipitated saccharide component.

2. The method of claim 1, further comprising redissolving water-soluble precipitated saccharide components to provide a saccharide solution.

3. The method of claim 1, further comprising adding an effective amount of hydrolyzing enzyme to the saccharide dispersion to hydrolyze a cellulose component thereof.

4. The method of claim 3, further comprising adding dilute acid.

5. The method of claim 3, wherein the hydrolyzing enzyme comprises cellulase.

6. The method of claim 3, further comprising fermenting the saccharide in the presence of a sugar-to-ethanol converting microorganism for a period of time and under suitable conditions for producing ethanol.

7. The method of claim 6, further comprising extracting the ethanol from the reaction mixture.

8. The method of claim 1 wherein the biomass is selected from the group consisting of hardwood, softwood, herbaceous plants, grasses, and agricultural residues.

9. The method of claim 1, wherein the organic solvent it selected from the group consisting of methanol, ethanol, n-propanol, isopropanol, acetone, and combinations thereof.

10. The method of claim 1, wherein the organic solvent is present in about a 2-100 fold volumetric excess relative to the volume of the biomass slurry.

11. A method for optimizing a pretreatment protocol for hydrolysis of lignocellulosic material, comprising:
pretreating a lignocellulosic material by an acid hydrolysis process to provide a pretreated material;
treating the pretreated material with a composition including an organic solvent to precipitate a saccharide component thereof; and
separating the saccharide component from the acidic solution.

12. The method of claim 11, wherein the lignocellulosic material used is selected from the group consisting of hardwood, softwood, herbaceous plants, grasses, and agricultural residues.

13. The method of claim 11, wherein the organic solvent it selected from the group consisting of methanol, ethanol, n-propanol, isopropanol, acetone, and combinations thereof.

14. The method of claim 11, wherein the organic solvent is present in about a 2-100 fold volumetric excess relative to the volume of the biomass slurry.

15. In a cellulose saccharification process, the improvement comprising:
precipitating a saccharide component of an acid treated lignocellulosic material by addition of an organic solvent to the reaction solution to facilitate separation of the saccharide component and the acid.

16. The process of claim 15, wherein the process is selected from a group consisting of making pulp, making paper, treating effluent from a pulp manufacturing process, treating effluent from a process of making paper, and combinations thereof.

17. The process of claim 15, wherein the process comprises a bioconversion process.
